(19) United States
(12) Patent Application Publication
(10) Pub. No.: US 2015/0148595 A1
(43) Pub. Date: May 28, 2015

(54) Method of Placing a Drainage Catheter System

(71) Applicant: AVENT, INC., Alpharetta, GA (US)

(72) Inventors: Alison S. Bagwell, Alpharetta, GA (US); Emily A. Reichart, Atlanta, GA (US)

(21) Appl. No.: 14/088,729

(22) Filed: Nov. 25, 2013

(51) Int. Cl. A61M 27/00; A61B 17/34

(52) U.S. Cl. CPC A61M 27/00; A61B 17/3403; A61B 17/3415
USPC 600/104; 604/543

(57) ABSTRACT

The present disclosure describes a single incision method of placing a drainage catheter in the peritoneal cavity.

This disclosure relates to apparatus or equipment for draining fluid from a body cavity and methods of using such apparatus or equipment to drain fluid from a body cavity. More specifically, the disclosure relates to equipment or apparatus used for paracentesis and methods of using such apparatus or equipment to perform paracentesis.

Clogging is a known problem for tubes used in drainage delivery systems, bowel management, nutrition, and dialysis. For example, many patients that require paracentesis procedures require them on a continuous basis and an indwelling catheter is placed for repeated access. Currently, these catheters are placed utilizing a tunneling technique. The tunneled catheters contain a polyester cuff at the most proximal end, to aid in promoting tissue in growth and act as a mechanical obstacle to bacterial infiltration. This cuff has been proven to be necessary to aid in cutting back on infection of the tract. However; the cuff makes it difficult to remove the catheter. When a tunneled catheter is clogged, kinked, or if the position of the tube is not allowing for proper drainage, there is a need to replace the catheter. When the catheter is damaged, it is necessary to remove the catheter and insert a new one. Replacing the catheter could cause additional trauma to the patient and the risk of increasing the probability of infection.

Drainage catheters are commonly placed utilizing a tunneling procedure. Using fluoroscopic guidance, a guide wire introducer with needle is inserted through the abdominal wall at the desired insertion site. The needle is removed while the guide wire introducer is left in place. An initial incision is made through the guide wire insertion site. A second incision is made 5-8 cm from the initial incision. A tunneler/catheter assembly is passed subcutaneously from the second incision down to and out through the incision at the guide wire insertion site until the polyester cuff on the catheter lies about 1 cm inside the second incision. The insertion site is dilated; a peel away introducer sheath is threaded over the guide wire and advanced into the peritoneal space. The guide wire and dilator are removed as a unit and the peel-away introducer sheath is left in place. The fenestrated end of the catheter is advanced into the sheath until all the fenestrations are within the peritoneal cavity. This can be verified under fluoroscopy as fenestrations are located along the barium sulfate stripe. The peel-away sheath is removed and the incision is closed at the insertion site. The catheter is then typically sutured to the skin superior to the second incision. Variations to the tunneling procedure can be performed (retrograde, antegrade, over the wire). However; all procedures utilize two incisions and the tunneling technique.

Infection is the most common complication associated with tunneled catheters. The distal end of the catheter is placed into the peritoneal cavity. Infection into the peritoneal cavity could lead to peritonitis which can result in patient death. Two incisions increase the risk of infection at the exit site.

### SUMMARY

The present disclosure addresses the problems described above by providing a method of placing a drainage catheter system. The method includes the steps of:

(1) inserting a needle through a side wall of a drainage catheter comprising an elongated tube having a substantially consistent cross-sectional size between a proximal end and a distal end, the tube having side walls defining a drainage lumen, the distal end of the tube being closed, and a plurality of holes in at least a portion of the side wall of the tube, the holes provided along a length of said tube defining an drainage section of the catheter;

(2) advancing the needle through the drainage lumen of the catheter until the needle penetrates beyond the closed distal end of the catheter to form a needle-catheter assembly; and

(3) inserting the needle-catheter assembly into the peritoneal cavity through the abdomen.

According to the method, the needle may be a safety needle. The step of inserting the needle-catheter assembly into the peritoneal cavity through the abdomen may be performed under fluoroscopy and/or performed with the use of a fiber optic eyepiece.

In an aspect of the method, the tip of the needle/catheter assembly may be tapered to provide dilation. Alternatively and/or additionally, the catheter may include a hydrophilic coating to assist with placement.

The method may further include the step of removing the needle through the drainage lumen of the catheter after the needle-catheter assembly reaches the peritoneal space and activating a retention element provided on the catheter.

The present disclosure may also encompass a steerable tissue tunneling device. The device includes a curved, bendable needle. The needle is desirably composed of a shape memory material. Exemplary materials include nitinol. The needle has a proximal portion and a distal portion and at least one preformed curvature along the distal portion. The preformed curvature is configured to reversibly flatten upon application of a straightening force. The device may also include a substantially straight cannula. The cannula is adapted to receive the needle such that a preformed curvature of the needle is reversibly flattened by inserting the needle into the cannula to form a coaxial assembly that restrains the needle in its flattened configuration. The coaxial assembly formed by inserting the needle into the cannula has a proximal portion and a distal portion.

According to the disclosure, the preformed curvature of the needle reverts (i.e., returns to its curved configuration) upon removal of the distal portion of the needle from the cannula. In an aspect of the disclosure, the needle may be further reversibly deformed to provide a curvature that is different from the preformed curvature. For example, a force may be applied to the needle to form a curvature that has a radius of curvature or a bend or shape that is different from the preformed curvature.

The device may further include an orientation indicator along the proximal end of the coaxial assembly to enable a user to identify the orientation of the preformed curvature of the needle. It is contemplated that the needle may further include a wire activated component to assist in obtaining the desired curvature upon removal of the cannula.

The present disclosure may also encompass a steerable tissue tunneling device composed of a curved, bendable hollow needle, the needle having a proximal portion and a distal portion and having at least one preformed curvature along the distal portion. The preformed curvature of the needle is configured to reversibly flatten upon application of a straightening force. The needle is desirably composed of a shape memory material. Exemplary materials include nitinol.

Other objects, advantages and applications of the present disclosure will be made clear by the following detailed description.

### BRIEF DESCRIPTION OF THE DRAWINGS

These and other features of this disclosure will be more readily understood from the following detailed description of the various aspects of the disclosure taken in conjunction with the accompanying drawings in which:

FIG. 1 is an illustration of features associated with a method of placing an exemplary drainage catheter system as a needle-catheter assembly.

FIG. 2 is an illustration of a needle that is inserted through the side wall of a catheter to make the needle-catheter assembly.

### DETAILED DESCRIPTION

Reference will now be made in detail to one or more embodiments, examples of which are illustrated in the drawings. It should be understood that features illustrated or described as part of one embodiment may be used with another embodiment to yield still a further embodiment. It is intended that the claims include these and other modifications and variations as coming within the scope and spirit of the disclosure.

The present disclosure addresses the problems described above by providing a method of placing a drainage catheter system. The method includes the steps of:

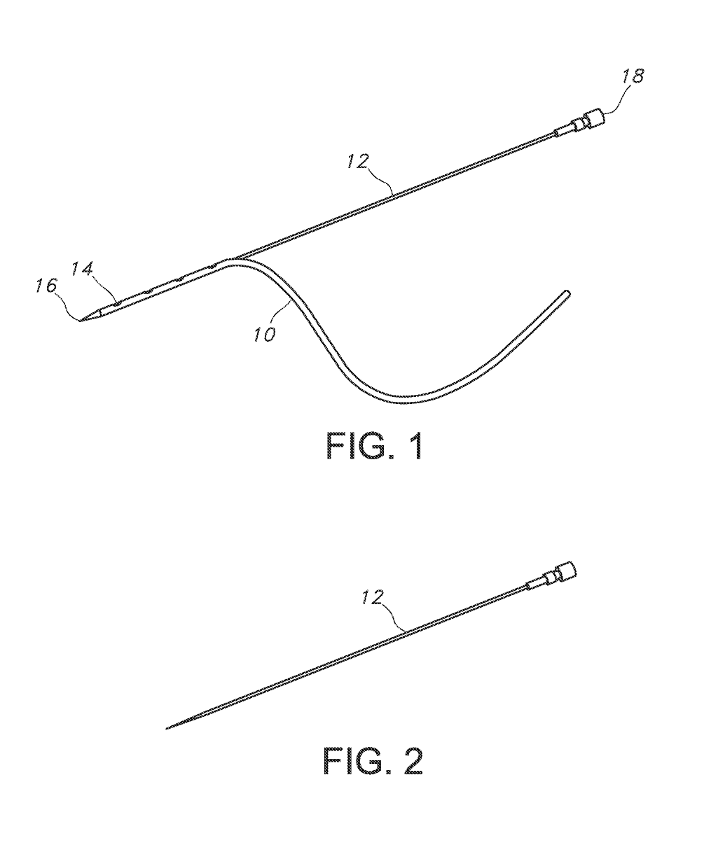

(1) inserting a needle 12 (FIG. 2) through a side wall of a drainage catheter 10 as generally illustrated in FIG. 1 in order to make a needle-catheter assembly, the catheter 10 comprising an elongated tube having a substantially consistent cross-sectional size between a proximal end and a distal end 16, the tube having side walls defining an internal drainage lumen, the distal end 16 of the tube being closed, and a plurality of holes 14 in at least a portion of the side wall of the tube, the holes provided along a length of the tube defining an drainage section of the catheter 10;

(2) advancing the needle 12 through the drainage lumen of the catheter 10 until the needle 12 penetrates beyond the closed distal end 16 of the catheter to form a needle-catheter assembly; and

(3) inserting the needle-catheter assembly into the peritoneal cavity through the abdomen.

According to the method, the needle 12 may be a safety needle. The step of inserting the needle-catheter assembly of FIG. 1 into the peritoneal cavity through the abdomen may be performed under fluoroscopy and/or performed with the use of a fiber optic eyepiece. The insertion of the needle-catheter assembly is done using a single incision, desirably made by the tip of the needle as the assembly is advanced through the abdomen.

In an aspect of the method, the tip of the needle-catheter assembly may be tapered to provide dilation. Alternatively and/or additionally, the catheter may include a hydrophilic coating to assist with placement.

The method may further include the step of removing the needle through the drainage lumen of the catheter after the needle-catheter assembly reaches the peritoneal space and activating a retention element provided on the catheter.

The present disclosure also encompasses a steerable tissue tunneling device. The device includes a curved, bendable needle. The needle is desirably composed of a shape memory material. Exemplary materials include nitinol. The needle may have a proximal portion and a distal portion and at least one preformed curvature along the distal portion. The preformed curvature is configured to reversibly flatten upon application of a straightening force.

While the present invention has been described in connection with certain preferred embodiments it is to be understood that the subject matter encompassed by way of the present disclosure is not to be limited to those specific embodiments. On the contrary, it is intended for the subject matter of the invention to include all alternatives, modifications and equivalents as can be included within the spirit and scope of the claims.

What is claimed is:

1. A method of placing a drainage catheter system, the method comprising:
inserting a needle through a side wall of a drainage catheter comprising an elongated tube and having a substantially consistent cross-sectional size between a proximal end and a distal end, the tube having side walls defining a drainage lumen, the distal end of the tube being closed, and a plurality of holes in at least a portion of the side wall of the tube, the holes provided along a length of said tube defining an drainage section of the catheter;
advancing the needle through the drainage lumen of the catheter until the needle penetrates beyond the closed distal end of the catheter to form a needle-catheter assembly; and
inserting the needle-catheter assembly into the peritoneal cavity through the abdomen.

2. The method of claim 1, wherein the needle is a safety needle,

3. The method of claim 1, wherein the step of inserting the needle-catheter assembly into the peritoneal cavity through the abdomen is performed under fluoroscopy and/or performed with the use of a fiber optic eyepiece.

4. The method of claim 1, wherein the tip of the needle/catheter assembly is tapered to provide dilation.

5. The method of claim 1, wherein the catheter includes a hydrophilic coating to assist with placement.

6. The method of claim 1, further comprising the step of removing the needle through the drainage lumen of the catheter after the needle-catheter assembly reaches the peritoneal space and activating a retention element provided on the catheter.

7. The device of claim 1 wherein a single incision is used to place the catheter in the peritoneal cavity.

8. The device of claim 6 further comprising an orientation indicator along the proximal end of the coaxial assembly to enable a user to identify the orientation of the preformed curvature of the needle.

9. The device of claim 8 wherein the needle may be reversibly deformed to provide a curvature that is different from the preformed curvature.

10. The device of claim 8 wherein the needle is composed of a shape memory material.

11. The device of claim 8 wherein the needle further comprises a wire activated component to assist in obtaining the desired curvature upon removal of the cannula.
